(19) United States
(12) Patent Application Publication
(10) Pub. No.: US 2010/0202583 A1
(43) Pub. Date: Aug. 12, 2010

(54) Systems and Methods for Exact or Approximate Cardiac Computed Tomography

(76) Inventors: Ge Wang, Blacksburg, VA (US); Hengyong Yu, Christiansburg, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/684,267

(22) Filed: Jan. 8, 2010

Related U.S. Application Data
(60) Provisional application No. 61/149,512, filed on Feb. 3, 2009.

(51) Int. Cl. A61B 6/03

(52) U.S. Cl. 378/9

(57) ABSTRACT

A computed tomography (CT) system has a composite scanning mode in which the x-ray focal spot undergoes a circular or more general motion in the vertical plane facing an object to be reconstructed. The x-ray source also rotates along a circular trajectory along a gantry encircling the object. In this way, a series of composite scanning modes are implemented, including a composite-circling scanning (CCS) mode in which the x-ray focal spot undergoes two circular motions: while the x-ray focal spot is rotated on a plane facing a short object to be reconstructed, the x-ray source is also rotated around the object on the gantry plane. In contrast to the saddle curve cone-beam scanning, the CCS mode requires that the x-ray focal spot undergo a circular motion in a plane facing the short object to be reconstructed, while the x-ray source is rotated in the gantry plane. Because of the symmetry of the mechanical rotations and the compatibility with the physiological conditions, this new CCS mode has significant advantages over the saddle curve from perspectives of both engineering implementation and clinical applications.

### STATEMENT OF GOVERNMENT INTEREST

This invention was partially supported by NIH grants EB002667, EB004287, and EB007288, under which the government may have certain rights.

### DESCRIPTION

### BACKGROUND OF THE INVENTION

1. Field of the Invention

The present invention generally relates to computed tomography (CT) and, more particularly, to a novel scanning mode, systems and methods for exact or approximate cardiac CT based on two composite x-ray focal spot rotations. The invention can be implemented on current cardiac CT scanners or built into upright CT scanners (with the Z-axis perpendicular to the earth surface). Beyond the CT field, the invention can also be applied to other imaging modalities such as x-ray phase-contrast tomography, positron emission tomography (PET), single photon emission computed tomography (SPECT), and so on. While the composite saddle-curve type scanning is performed, the patient/object can also be constantly or adaptively translated to enrich the family of trajectories.

2. Background Description

Since its introduction in 1973 (see, Hounsfield, “Computerized transverse axial scanning (tomography): Part I. Description of system”, British Journal of Radiology, 1973, 46: pp. 1016-1022), x-ray CT has revolutionized clinical imaging and become a cornerstone of radiology departments. Closely correlated to the development of x-ray CT, the research for better image quality at lower dose has been pursued for important medical applications with cardiac CT being the most challenging example. The first dynamic CT system is the Dynamic Spatial Reconstructor (DSR) built at the Mayo Clinic in 1979 (see, Robb, R. A., et al., “High-speed three-dimensional x-ray computed tomography: The dynamic spatial reconstructor”, Proceedings of the IEEE, 1983, and Ritman, R. A. Robb. and L. D. Harris, “Imaging physiological functions: experience with the DSR”, 1985: philadelphia: praeger). In a 1991 SPIE conference, for the first time we presented a spiral cone-beam scanning mode to solve the long object problem (see Wang, G., et al., “Scanning cone-beam reconstruction algorithms for x-ray microtomography”, SPIE, vol. 1556, pp. 99-112, 1991, and Wang, G., et al., “A general cone-beam reconstruction algorithm”, IEEE Trans. Med. Imaging, 1993. 12: pp. 483-496) (reconstruction of a long object from longitudinally truncated cone-beam data). In the 1990s, single-slice spiral CT became the standard scanning mode of clinical CT (see Kalender, W. A., “Thin-section three-dimensional spiral CT: is isotropic imaging possible?”, Radiology, 1995, 197(3): pp. 578-80). In 1998, multi-slice spiral CT entered the market (see Taguchi, K. and H. Aradate, “Algorithm for image reconstruction in multi-slice helical CT”, Med. Phys., 1998. 25(4): pp. 550-561, and Kachelriess, M., S. Schaller, and W. A. Kalender, “Advanced single-slice rebinning in cone-beam spiral CT”, Med. Phys., 2000, 27(4): pp. 754-772). With the fast evolution of the technology, helical cone-beam CT becomes the next generation of clinical CT.

Moreover, just as there have been strong needs for clinical imaging, there are equally strong demands for pre-clinical imaging, especially of genetically engineered mice (see Holdsworth, D. W., “Micro-CT in small animal and specimen imaging”, Trends in Biotechnology, 2002, 20(8): pp. S34-S39, Paulus, M. J., “A review of high-resolution X-ray computed tomography and other imaging modalities for small animal research”, Lab. Animal, 2001, 30: pp. 36-45, and Wang, G., “Micro-CT scanners for biomedical applications: an overview”, Adv. Imaging, 2001, 16: pp. 18-27). Although there has been an explosive growth in the development of cone-beam micro-CT scanners for small animal studies, the efforts are generally limited to high spatial resolution of 20-100 μm at large radiation dose (see again, Wang, G., “Micro-CT scanners for biomedical applications: an overview”, supra). To meet the clinical needs and technical challenges, it is imperative that cone-beam CT methods and architectures must be developed in a systematic and innovative manner so that the momentum of the CT technical development, clinical and pre-clinical applications can be sustained and increased. Hence, our CT research has been for superior dynamic volumetric low-dose imaging capabilities. Since the long object problem has been well studied by now, we recently started working on the quasi-short object problem (reconstruction of a short portion of a long object from longitudinally truncated cone-beam data involving the short object).

Currently, the state-of-the-art cone-beam scanning for clinical cardiac imaging follows either circular or helical trajectories. The former only permits approximate cone-beam reconstruction because of the inherent data incompleteness. The latter allows theoretically exact reconstruction but due to the openness of helical scanning there is no ideal scheme to utilize cone-beam data collected near the two ends of the involved helical segment. Recently, saddle-curve cone-beam scanning was studied for cardiac CT (see Pack, J. D., F. Noo, and R Kudo, “Investigation of saddle trajectories for cardiac CT imaging in cone-beam geometry”, Phys Med Biol, 2004, 49(11): pp. 2317-36, and Yu, H. Y., et al., “Exact BPF and FBP algorithms for nonstandard saddle curves”, Medical Physics, 2005, 32(11): pp. 3305-3312), which can be directly implemented by compositing circular and linear motions: while the x-ray source is rotated in the vertical x-y plane, it is also driven back and forth along the z-axis. Because the electro-mechanical needs for converting a motor rotation to the linear oscillation and handling the acceleration of the x-ray source along the z-axis, it is a major challenge to implement directly the saddle-curve scanning mode in practice, and it has not been employed by any CT company. However, it does represent a very promising solution to the quasi-short object problem.

### SUMMARY OF THE INVENTION

It is therefore an object of the present invention to provide a novel scanning mode, system design and associated methods for cardiac imaging and other applications which solve the quasi-short object problem, which is the reconstruction of a short portion of a long object from longitudinally truncated cone-beam data involving the short object.

According to the invention, there is provided a CT system in which the x-ray focal spot undergoes a circular or more general motion in the plane facing an object (heart) to be reconstructed, the x-ray source also rotates along a circular trajectory along the gantry in the gantry plane. Thus, the invention implements a series of composite scanning modes, including composite-circling scanning (CCS) mode in which the x-ray focal spot undergoes two circular motions: while the x-ray focal spot is rotated on a plane facing a short object to be reconstructed, the x-ray source is also rotated around the object on the gantry plane. In contrast to the saddle curve cone-beam scanning, the CCS mode of the invention requires that the x-ray focal spot undergo a circular motion in a plane facing the short object to be reconstructed, while the x-ray source is rotated in the gantry plane. Because of the symmetry of the mechanical rotations and the compatibility with the physiological conditions, this new CCS mode has significant advantages over the saddle curve from perspectives of both engineering implementation and clinical applications. The generalized backprojection filtration (BPF) method is used to reconstruct images from data collected along a CCS trajectory within a planar detector.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 is a pictorial representation of a CT apparatus of the type which may be used to implement the invention;

FIG. 2 is a diagrammatic illustration of the CCS mode according to the invention as applied to cardiac imaging;

FIGS. 3A to 3D are graphical representations of composite-circling curves with different parameter combinations;

FIG. 4 is a diagrammatic illustration, similar to FIG. 2, showing that the saddle-curve-like trajectory can be further enriched by performing a constant or adaptive table translation simultaneously;

FIG. 5 is an illustration of the composite-circling scanning mode according to the invention;

FIG. 6 is a graphical representation of the PI-Segment (chord) and associate PI-arc;

FIG. 7 is a diagram showing the local coordinate system with the composite-circling scanning trajectory according to the invention;

FIG. 8 is a projection of the chord and composite-circling trajectory on the x-y plane;

FIGS. 9A to 9D are illustrations of reconstructed slices of the 3D Shepp-Logan phantom in the natural coordinate system with the display window; and

FIGS. 10A to 10D are illustrations similar to FIGS. 7A to 7D but from noisy data with N₀=10⁶.

### DETAILED DESCRIPTION OF A PREFERRED EMBODIMENT OF THE INVENTION

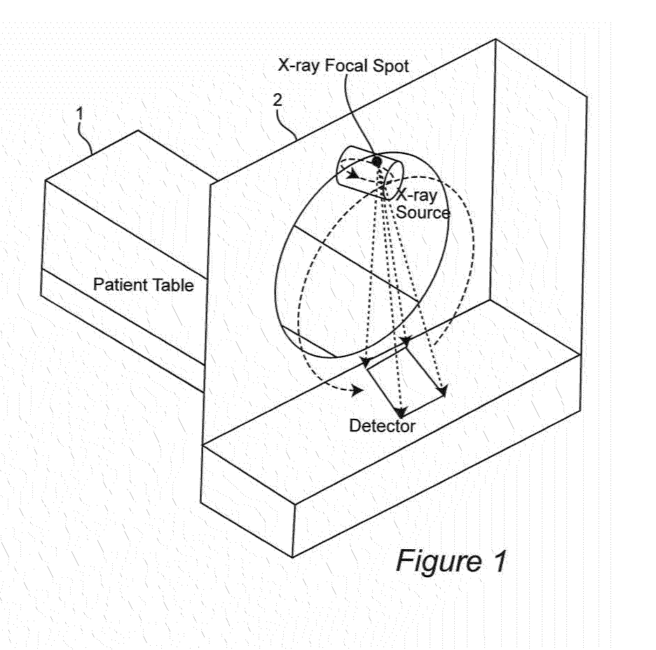

Referring now to the drawings, and more particularly to FIG. 1, there is shown an example of a CT apparatus which includes a sliding table 1 one which a patient may be placed so as to pass within a gantry 2 on which an x-ray source (not shown) is mounted to rotate around the patient. X-ray sensors (not shown) are positioned on the opposite side of the gantry from the x-ray source. Data scans are mathematically combined by a computer (not shown) to generate a tomographic reconstruction of the object (e.g., the patient's heart) being examined.

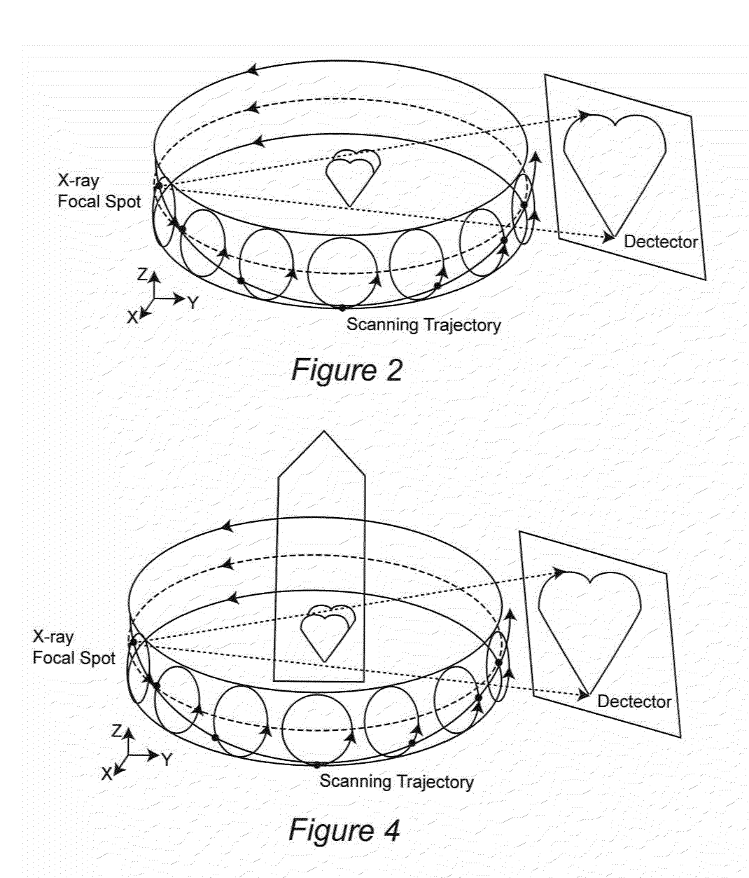

We have invented a composite-circling scanning (CCS) mode to solve the quasi-short object problem. Our goal is to enlarge the space of candidate scanning curves into a family of saddle-like curves for determination of the optimal solution to the quasi-short object problem. FIG. 2 illustrates the CCS mode according to our invention as applied to cardiac imaging. In the systems and methods implementing the CCS mode, the trajectory is a composition of two circular motions. While the x-ray focal spot undergoes a circular motion on a vertical plane facing an object to be reconstructed, the x-ray source is also rotated around the object along a circular trajectory in the horizontal plane. Once the necessary projection datasets are acquired, exact reconstruction can be obtained.

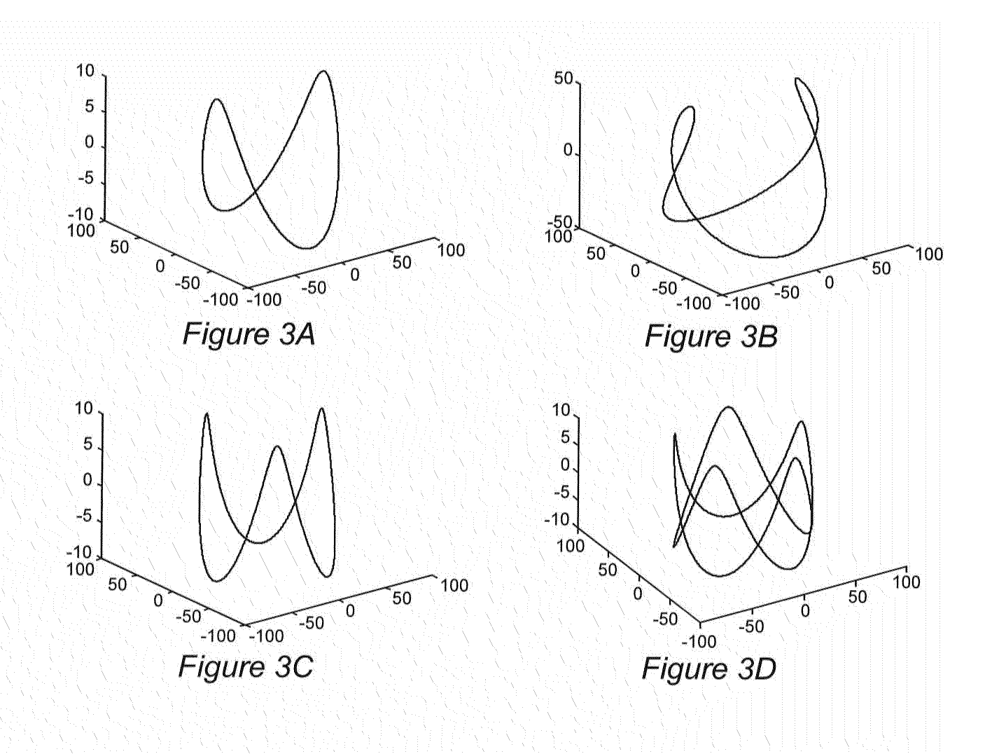

When an x-ray focal spot is in a 2D (no, linear, circular, or other) motion on the plane (or more general in a 3D motion within the neighborhood) facing a short object to be reconstructed, and the x-ray source is at the same time rotated in a transverse plane of a patient, the synthesized 3D scanning trajectory with respect to the short object can be a circle, a saddle curve, a CCS trajectory, or other interesting loci. Let R₁ₐ≧0 and R_{1b}≧0 the lengths of the two semi-axes of the scanning range in the focal plane facing the short object, and R₂>0 the radius of the tube scanning circle on the x-y plane, we mathematically define a family of saddle-like composite trajectory as:

$$Γ = \{ ρ ( s ) \vert \begin{matrix}{ρ}_{1} ( s ) = {R}_{2} cos ( {ω}_{2} s ) - {R}_{1 b} sin ( {ω}_{1} s ) sin ( {ω}_{2} s ) \\ {ρ}_{2} ( s ) = {R}_{2} sin ( {ω}_{2} s ) + {R}_{1 b} sin ( {ω}_{1} s ) cos ( {ω}_{2} s ) \\ {ρ}_{3} ( s ) = {R}_{1 a} cos ( {ω}_{1} s ) ,\end{matrix} \} , \qquad ( 1 )$$

where s ∈ [symbol] represents a real time parameter, and ω₁ and ω₂ are the angular frequencies of the focal spot and tube rotations, respectively. When the ratio between ω₁ and ω₂ is an irrational number or a rational number with large numerator in its reduced form, the scanning curve covers a band of width 2R₁ₐ, allowing a rather uniform sampling pattern. With all the possible settings of R₁ₐ, R_{1b}, R₂, ω₁ and ω₂, we have a family of cone-beam scanning trajectories including saddle curve and CCS loci that can be used to solve the quasi-short problem exactly. However, we are particularly interested in a rational ratio between ω₁ and ω₂ in this paper, which will results a periodical scanning. Without loss of generality, we re-express Eq.(1) as

$$Γ = \{ ρ ( s ) \vert \begin{matrix}{ρ}_{1} ( s ) = {R}_{2} cos ( s ) - {R}_{1 b} sin ( ms ) sin ( s ) \\ {ρ}_{2} ( s ) = {R}_{2} sin ( s ) + {R}_{1 b} sin ( ms ) cos ( s ) \\ {ρ}_{3} ( s ) = {R}_{1 a} cos ( ms )\end{matrix} \} , \qquad ( 2 )$$

where m>1 is a rational number. When R_{1b}=0 and m=2, we obtain the standard saddle curve. When R₁ₐ=R_{1b}, we have our CCS trajectory. Some representative CCS curves are shown in FIGS. 3A to 3D. The parameter combinations for FIGS. 3A to 3D are as follows:

FIG. 3A: m=2, R1a=R1b=10, R2=57

FIG. 3B: m=2, R1a=R1b=50, R2=57

FIG. 3C: m=3, R1a=R1b=10, R2=57

FIG. 3D: m=2.5, R1a=R1b=10, R2=57

The saddle-curve-like trajectories of FIGS. 3A to 3D can be further enriched by performing a constant or adaptive table translation simultaneously, as illustrated in FIG. 4.

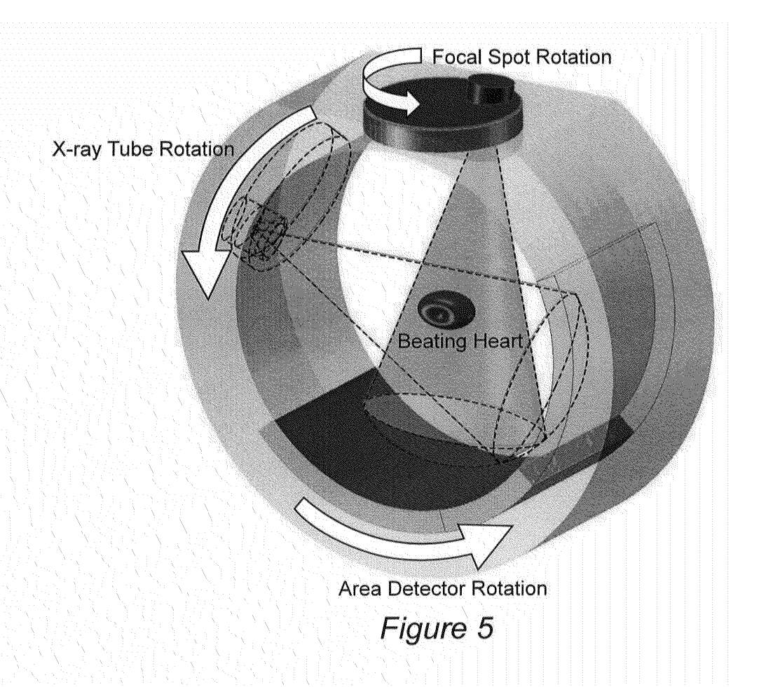

As mentioned above, while the saddle curve cone-beam scanning does meet the requirement for exact cone-beam cardiac CT, it imposes quite difficult mechanical constraints. In contrast to the saddle curve cone-beam scanning, our proposed CCS requires that the x-ray focal spot undergo a circular motion in a plane facing the short object to be reconstructed, while the x-ray source is rotated in the x-y plane, as shown in FIG. 5.

In the CT system shown in FIG. 5, the scanning trajectory is a composition of two circular motions. While an x-ray focal spot is rotated in a plane facing a short object to be reconstructed, the x-ray source is also rotated around the object on the gantry plane. Once a projection dataset is acquired, exact or approximate reconstruction can be done in a number of ways.

Preferably, we may let the patient sit or stand straightly and make the x-y plane parallel to the earth surface. Because of the symmetry of the proposed mechanical rotations and the compatibility with the physiological conditions, this approach to cone-beam CT has significant advantages over the existing cardiac CT scanners and the standard saddle curve oriented systems from perspectives of both engineering implementation and clinical applications.

### Exact Reconstruction Method

Assume an object function ƒ(r) is located at the origin of the natural coordinate system O. For any unit vector β, let us define a cone-beam projection of ƒ(r) from a source point ρ(s) on a CCS trajectory by

$${D}_{f} ( ρ ( s ) , β ) := {\int }_{0}^{∞} f ( ρ ( s ) + t β ) d t \cdot \qquad ( 3 )$$

Then, we define the unit vector p as the one pointing to r from ρ(s) on the CCS trajectory:

$$β ( r , s ) := \frac{r - ρ ( s )}{\vert r - ρ ( s ) \vert} , \qquad ( 4 )$$

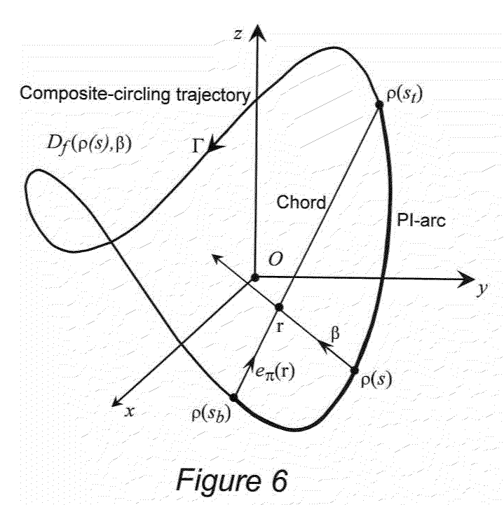

As shown in FIG. 6, a generalized PI-line can be defined as the line passing that point and intersecting the CCS trajectory at two points ρ(s_{b}(r)) and ρ(sₜ(r)), where s_{b}=s_{b}(r) and sₜ=sₜ(r) are the rotation angles corresponding to these two points. At the same time, the PI-segment (also called chord) is defined as the part of the PI-line between ρ(s_{b}(r)) and ρ(sₜ(r)), the PI-arc is defined as the part of the scanning trajectory between ρ(s_{b}(r)) and ρ(sₜ(r)), and the PI-interval as (s_{b},sₜ). All the PI-segments form a convex hull H of the CCS curve where the exact reconstruction is achievable. Note that the uniqueness of the chord is not required. We also need a unit vector along the chord:

$${e}_{π} ( r ) := \frac{ρ ( {s}_{t} ( r ) ) - ρ ( {s}_{b} ( r ) )}{\vert ρ ( {s}_{t} ( r ) ) - ρ ( {s}_{b} ( r ) ) \vert} \cdot \qquad ( 5 )$$

To perform exact reconstruction from the data collected along a CCS trajectory, we need to setup a local coordinate system. Initially, we only consider the circling scanning trajectory \tilde{Γ} of the x-ray tube in the x-y plane which can be expressed as

\tilde{Γ}={\tilde{ρ}(s)|\tilde{ρ}₁(s)=R₂ cos (s),\tilde{ρ}₂(s)=R₂ sin(s),\tilde{ρ}₃(s)=0}. (6)

For a given s, we define a local coordinate system for \tilde{ρ}(s) by the three orthogonal unit vectors:

d₁:=(−sin(s),cos(s),0),d₂:=(0,0,1) and d₃:=(−cos(s),−sin(s),0),

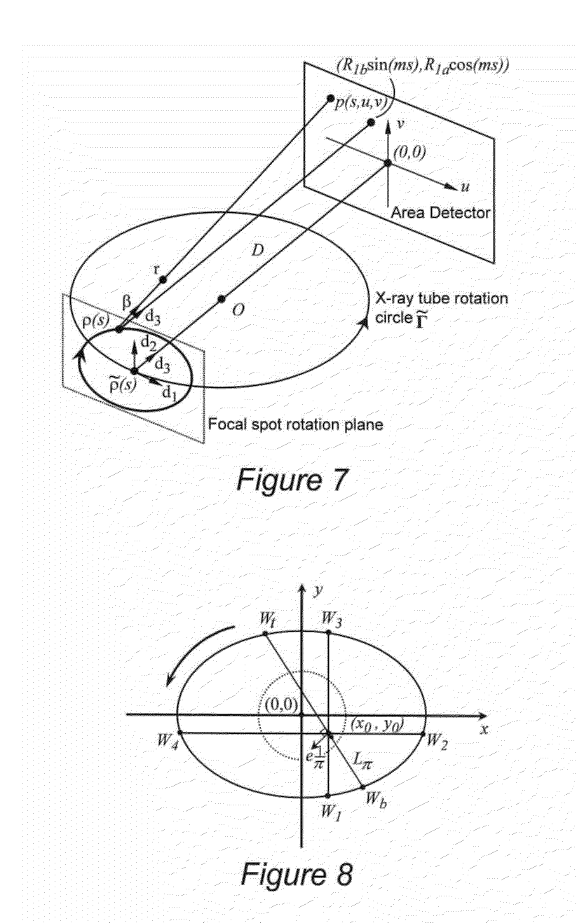

as shown in FIG. 7. Equispatial cone-beam data are measured on a planar detector array parallel to d₁ and d₂ at a distance D from \tilde{ρ}(s) with D=R₂+D_{c}, where the constant D_{c} is the distance between the z-axis and the detector plane. A detector position in the array is denoted by (u,v), which are signed distances along d₁ and d₂ respectively. Let (u,v)=(0, 0) correspond to the orthogonal projection of \tilde{ρ}(s) onto the detector array. If s is given, (u,v) are determined by β. Thus, the cone-beam projection data of a direction βfrom \tilde{ρ}(s) can be re-written in the planar detector coordinate system as \tilde{ρ}(s,u,v):=D_{ƒ}(\tilde{ρ}(s),β) with

$$u = \frac{D β \cdot {d}_{1}}{β \cdot {d}_{3}} , v = \frac{D β \cdot {d}_{2}}{β \cdot {d}_{3}} \cdot \qquad ( 7 )$$

Now, let us consider the circle rotation of the focal spot at the given time s. According to our definition of Eq.(2), the focal spot rotation plane is parallel to the local area detector. And the orthogonal projection of the composite-circling focal spot position ρ(s) in the above mentioned local area detector is (R_{1b} sin(ms),R₁ₐ cos(ms)). Finally, the cone-beam projection data of a direction β from ρ(s) can be re-written in the same local planar detector coordinate system as p(s,u,v):=D_{ƒ}(ρ(s),β) with

$$u = \frac{D β \cdot {d}_{1}}{β \cdot {d}_{3}} + {R}_{1 b} sin ( ms ) , v = \frac{D β \cdot {d}_{2}}{β \cdot {d}_{3}} + {R}_{1 a} cos ( ms ) \cdot \qquad ( 8 )$$

### Reconstruction Steps

In 2002, an exact and efficient helical cone-beam reconstruction method was developed by Katsevich (see Katsevich, A., “Theoretically exact filtered backprojection-type inversion algorithm for spiral CT”, SIAM J. Appl. Math., 2002, 62(6): pp. 2012-2026, and Katsevich, A., “An improved exact filtered backprojection algorithm for spiral computed tomography”, Advances in Applied Mathematics, 2004, 32(4): pp. 681-697), which is a significant breakthrough in the area of helical/spiral cone-beam CT. The Katsevich formula is in a filtered-backprojection (FBP) format using data from a PI-arc based on the so-called PI-Segment and the Tam-Danielsson window. By interchanging the order of the Hilbert filtering and backprojection, Zou and Pan proposed a backprojection filtration (BPF) formula in the standard helical scanning case (see Zou, Y. and X. C. Pan, “Exact image reconstruction on PI-lines from minimum data in helical cone-beam CT”, Physics in Medicine and Biology, 2004, 49(6): pp. 941-959). This BPF formula can reconstruct an object only from the data in the Tam-Danielsson window. For important biomedical applications including bolus-chasing CT angiography (see Wang, G. and M. W. Vannier, “Bolus-chasing angiography with adaptive real-time computed tomography”, U.S. Pat. No. 6,535,821) and electron-beam CT/micro-CT, our group contributed the first proof of the general validities for both the BPF and FBP formulae in the case of cone-beam scanning along a general smooth scanning trajectory (see Ye, Y., et al. “Exact reconstruction for cone-beam scanning along nonstandard spirals and other curves”, Developments in X-Ray Tomography IV, Proceedings of SPIE, 5535:293-300, Aug. 4-6, 2004. Denver, Colo., United States, Ye, Y. B., et al., “A general exact reconstruction for cone-beam CT via backprojection-filtration,” IEEE Transactions on Medical Imaging, 2005, 24(9): pp. 1190-1198,Ye, Y. B. and G. Wang, “Filtered backprojection formula for exact image reconstruction from cone-beam data along a general scanning curve”, Medical Physics, 2005, 32(1): pp. 42-48, and Zhao, S. Y., H. Y. Yu, and G. Wang, “A unified framework for exact cone-beam reconstruction formulas”, Medical Physics, 2005, 32(6): pp. 1712-1721. Our group also formulated the generalized FBP and BPF algorithms in a unified framework, and applied them into the cases of generalized n-PI-window geometry (see Yu, H. Y., et al., “A backprojection-filtration algorithm for nonstandard spiral cone-beam CT with an n-PI-window”, Physics in Medicine and Biology, 2005, 50(9): pp. 2099-2111) and saddle curves. Noting that our general BPF and FBP formulae are valid to any smooth scanning loci, they can be applied to the reconstruction problem of the CCS trajectory. Based on our experiences of the reconstruction problem of the saddle curves, the BPF algorithm is more computational efficient than FBP, and they have similar noise characteristics. Therefore, we will only focus on the BPF method and describe its major steps as the following.

### Step 1. Cone-Beam Data Differentiation

For every projection, compute the derivative data G(s,u,v) from the projection data p(s,u,v):

$$\begin{matrix}\begin{matrix}{G ( s , u , v ) ≡ \frac{\partial }{\partial s} {D}_{f} ( ρ ( s ) , β ) \vert}_{β fixed} \\ = \frac{d}{d s} ρ ( s , u , v ) {\vert}_{β fixed} \\ = ( \frac{\partial }{\partial s} + \frac{\partial u}{\partial s} \frac{\partial }{\partial u} + \frac{\partial v}{\partial s} \frac{\partial }{\partial v} ) ρ ( s , u , v )\end{matrix} \\ where & ( 9 ) \\ \frac{\partial u}{\partial s} = \frac{{( u - {R}_{1 b} sin ( ms ) )}^{2}}{D} + D + {mR}_{1 b} cos ( ms ) , & ( 10 ) \\ \frac{\partial v}{\partial s} = \frac{( u - {R}_{1 b} sin ( ms ) ) ( v - {R}_{1 a} cos ( ms ) )}{D} - {mR}_{1 a} sin ( ms ) \cdot & ( 11 )\end{matrix}$$

The detail derivatives of Eqs. (10-11) are in the appendix A.

### Step 2. Weighted Backprojection

For every chord specified by s_{b} and sₜ, and for every point r on the chord, compute the weighted backprojection data:

$$\begin{matrix}b ( r ) := {\int }_{{s}_{b} ( r )}^{{s}_{t} ( r )} G ( s , \overline{u} , \overline{v} ) \frac{d s}{\vert r - ρ ( s ) \vert} , \\ with & ( 12 ) \\ \overline{u} = \frac{D β ( r , s ) \cdot {d}_{1}}{β \cdot {d}_{3}} + {R}_{1 b} sin ( ms ) , \\ v = \frac{D β ( r , s ) \cdot {d}_{2}}{β \cdot {d}_{3}} + {R}_{1 a} cos ( ms ) \cdot & ( 13\end{matrix}$$

### Step 3. Inverse Hilbert Filtering

For every chord specified by s_{b} and sₜ perform the inverse Hilbert filtering along the 1D chord direction eₐ(r) to reconstruct ƒ(r) from b(r). The filtering method and formula are the same as our previous papers (see Yu, H. Y., et al., “Exact BPF and FBP algorithms for nonstandard saddle curves”, Medical Physics, 2005, 32(11): pp. 3305-3312, Ye, Y. B., et al., “A general exact reconstruction for cone-beam CT via backprojection-filtration”, IEEE Transactions on Medical Imaging, 2005, 24(9): pp. 1190-1198, and Yu, H. Y., et al., “A backprojection-filtration algorithm for nonstandard spiral cone-beam CT with an n-PI-window”, Physics in Medicine and Biology, 2005, 50(9): pp. 2099-2111).

### Step 4. Image Rebinning

Rebin the reconstructed image into the natural coordinate system by determining the chord(s) for each grid point in the natural coordinate system. The rebinning scheme is the same as what we did for the saddle curve (see Yu, H. Y., et al., “Exact BPF and FBP algorithms for nonstandard saddle curves”, Medical Physics, 2005, 32(11): pp. 3305-3312). However, there are some differences to numerically determining a chord, which will be detailed in the next subsection.

### Chord Determination

For our CCS mode, we assume that R_{1b}≧R₂/(2m) . In this case, the projection of the trajectory in the x-y plane will be a convex single curve (see appendix B). Among the all the potential CCS modes, we initially study the case m=2 which is similar to a saddle curve. Hence, we will study how to determine a chord for a fixed point for m=2 in this subsection.

As shown in FIG. 8, to find a chord containing the fixed point r₀=(x₀,y₀,z₀) in the convex hull H, we first consider the projection curve of the trajectory in x-y plane. Due to the convexity of the projection curve, any line passing a point inside the curve in the x-y plane has two and only two intersections with the projection curve. Then, we consider a special plane x=x₀. In this case, there are two intersection points between the plane and the projection curve (CCS trajectory). Solving the equation R₂ cos(s)−R_{1b} sin(2s)sin(s)=x₀, that is, R₂ cos(s)−2R_{1b}(1−cos²(s))cos(s)=x₀, we can obtain one and only one real root −1≦q_{cos}≦1 for cos(s) (see King, B., ed. “Beyond the Quartic Equation”, 1996: Boston, Mass.), and the view angles sₜ=−cos⁻¹(q_{cos}) and s₃=−s₁ that correspond to the two intersection points W₁ and W₃. On the other hand, we consider another special plane y=y₀. Solving the equation R₂ sin(s)+R_{1b} sin(2s)cos(s)=y₀, that is R₂ sin(s)+2R_{1b}(1−sin²(s))sin(s)=y₀, we have the only real root −1≦qₛᵢₙ≦1 and corresponding to the two intersection points W₂ and W₄. Obviously, the above four angles satisfy s₁<s₂<s₃<s₄. Now, we consider a chord L_{z} intersecting with the line L_{z} parallel to the z-axis and containing the point (x₀,y₀,z₀). In the x-y plane, the projection of the line is the point (x₀,y₀) and the projection of L_{z} passes through the point (x₀,y₀). According to the definition of a CCS curve, the line W̅₁̅W̅₃̅ intersects L_{z} at (x₀,y₀,R₁ₐ cos(2s₁)), while W̅₂̅W̅₄̅ intersects L_{z} at (x₀,y₀,R₁ₐ cos(2s₂)). Recall that we have assumed that r₀ is inside the convex hull H, there will be R₁ₐ cos(2s₁)≦z₀≦R₁ₐ cos(2s₂), that is, R₁ₐ(2q_{cos}²−1)≦z₀≦R₁ₐ(1−2qₛᵢₙ²). When the starting point W_{b} of L_{z} moves from W₁ to W₂ smoothly, the corresponding end point W₁ will change from W₃ to W₄ smoothly, and the z-coordinate of its intersection with L_{z} will vary from R₁ₐ(2q_{cos}²−1) to R₁ₐ(1−2qₛᵢₙ²) continuously. Therefore, there exists at least one chord L_{z} that intersects L_{z} at r₀ and satisfies s_{b1} ∈(s₁,s₂), sₜ₁ ∈(s₃,s₄). Because the CCS trajectory is closed, we can immediately obtain another chord corresponding to the PI-interval (sₜ₁,s_{b1}+2/π). The union of the two intervals yields a 2π scan range. Similarly, we can find s_{b2} ∈(s₂,s₃) and sₜ₂ ∈(s₄,s₁+2π) as well as the chord intervals (s_{b2},sₜ₂) and (sₜ₂,s_{b2}+2π). Hence, we can perform reconstruction at least four times for a given point inside the hull of a CCS trajectory.

Based on the above discussion, to illustrate the procedure of chord determination, we numerically find the chord corresponding to the P1-interval (s_{b1},sₜ₁) by the following pseudo-codes.
S1: Set s_{b min}=s₁,s_{b max}=s₂;
S2: Set s_{b1}=(s_{b max}+s_{b min})/2 and find sₜ₁ ∈(s₃,s₄) so that ρ̅(̅s̅_̅{̅b̅1̅}̅)̅ρ̅(̅s̅ₜ̅₁̅)̅ ρ̅(̅s̅_̅{̅b̅1̅}̅)̅ρ̅(̅s̅ₜ̅₁̅)̅ intersects L_{z}:
   S2.1 Compute the unit direction e_{π}^{L} in the X-Y plane (see FIG. 5);
   S2.2: Set s_{t min}=s₃, s_{t max}=s₄, and sₜ₁=(s_{t max}+s_{t min})/2;
   S2.3: Compute the projection δ=(ρ(sₜ₁)−r₀)e_{π}¹⁹⁵;
   S2.4: If δ=0 stop, else go to S2.2 and set s_{t max}=sₜ₁ if δ<0; and set s_{t min}=sₜ₁ if δ<0;
S3: Compute z′ of the intersection point between ρ̅(̅s̅_̅{̅b̅1̅}̅)̅ρ̅(̅s̅ₜ̅₁̅)̅ ρ̅(̅s̅_̅{̅b̅1̅}̅)̅ρ̅(̅s̅ₜ̅₁̅)̅ and L_{z};
S4: If z′=z₀ stop, else go to S2 and set s_{b max}=s_{b1} if z′>z₀ and set s_{b min}=s_{b1} z′<z₀.
   Given numerically implementation details and tricks of the above BPF method and chord determination are similar to what we have disclosed in our previous works, here we will not repeat them.

### Simulation Results

To demonstrate the merits of the CCS mode and validate the correctness of the exact reconstruction method, we implemented the reconstruction procedure in MatLab on a PC (2.0 Gagabyte memory, 2.8 G Hz CPU), with all the computationally intensive parts coded in C. A CCS trajectory was assumed with R₁ₐ=R_{1b}=10 cm, R₂=57 cm and m=2.0, which is consistent with the available commercial CT scanner and satisfied the requirements of the exact reconstruction of a quasi-short object, such as a head and heart. In our simulation, the well known 3D Shepp-Logan head phantom (see Shepp, L. A. and B. F. Logan, “The Fourier Reconstruction of a Head Section”, IEEE Transactions on Nuclear Science, 1974, NS21(3): pp. 21-34) was used. And the phantom was contained in a spherical region whose radius is 10 cm. We also assumed a virtual plane detector and set the distance from the detector array to the z-axis (D₀) to zero. The detector array included 523×732 detector elements with each covering 0.391×0.391 mm². When the X-ray source was moved along the CCS trajectory a turn, 1200 cone-beam projections were equi-angularly acquired.

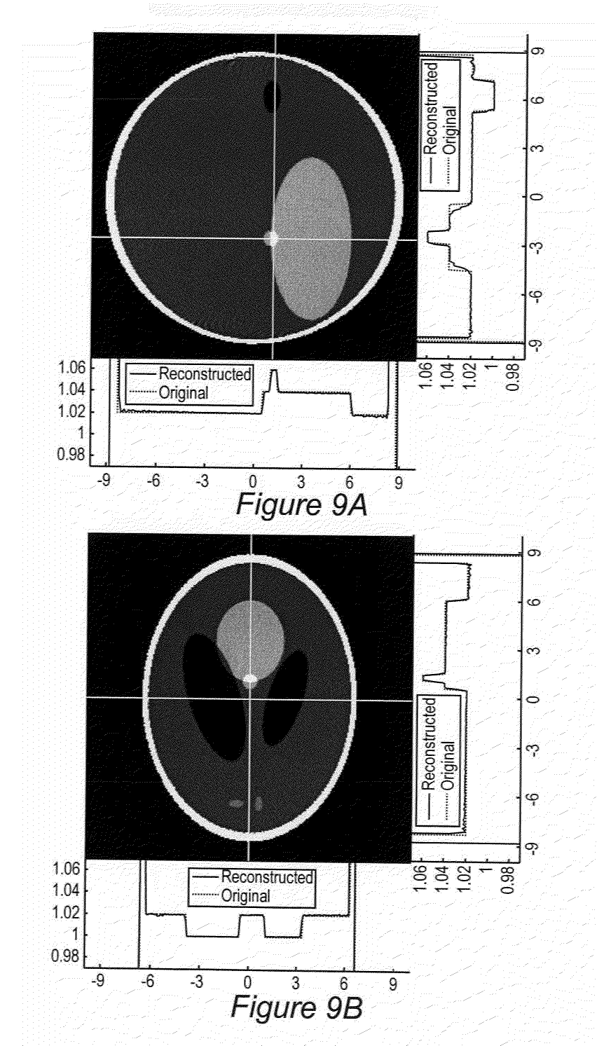

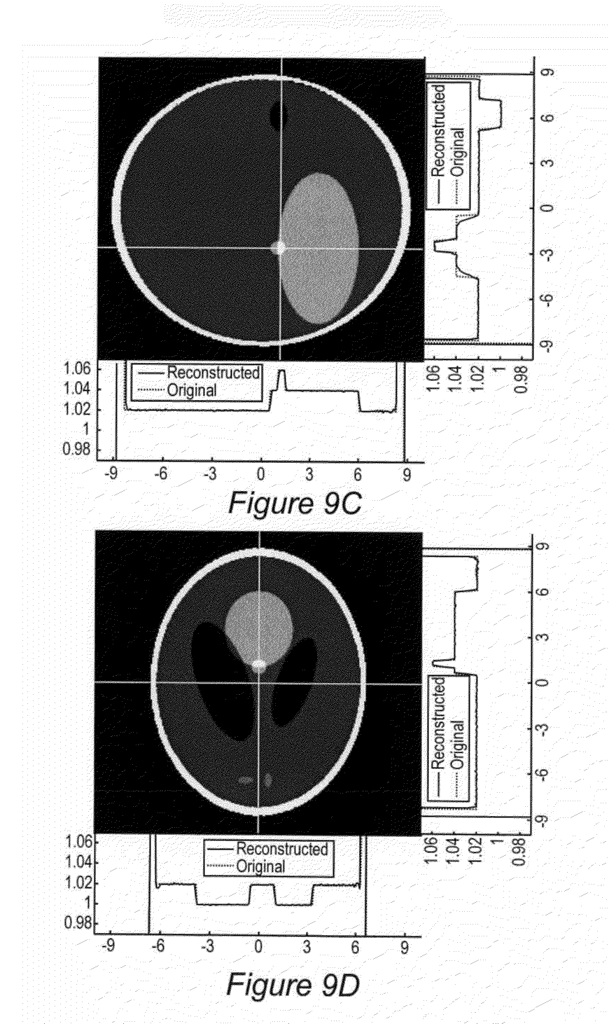

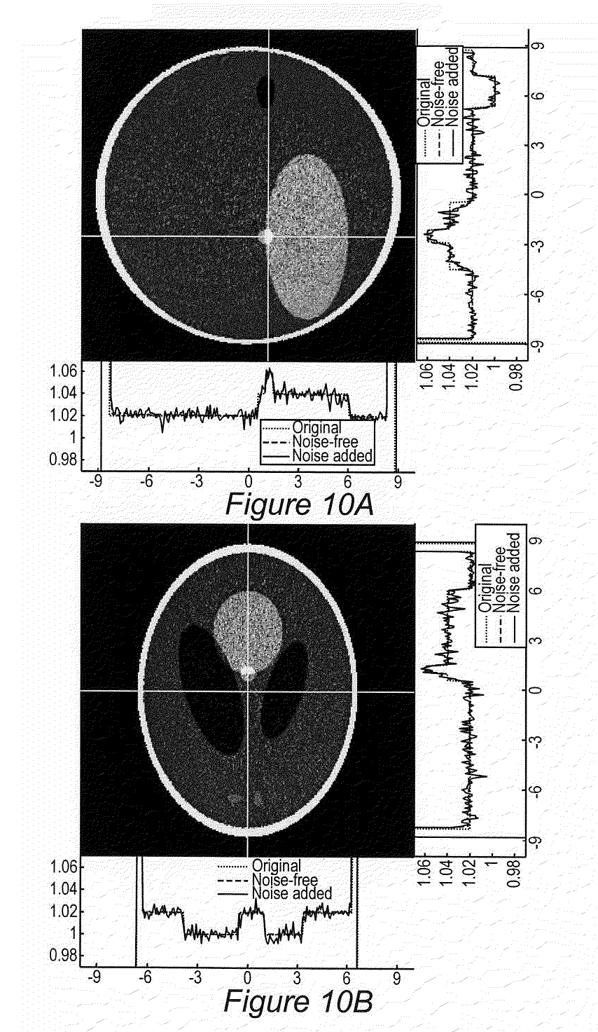

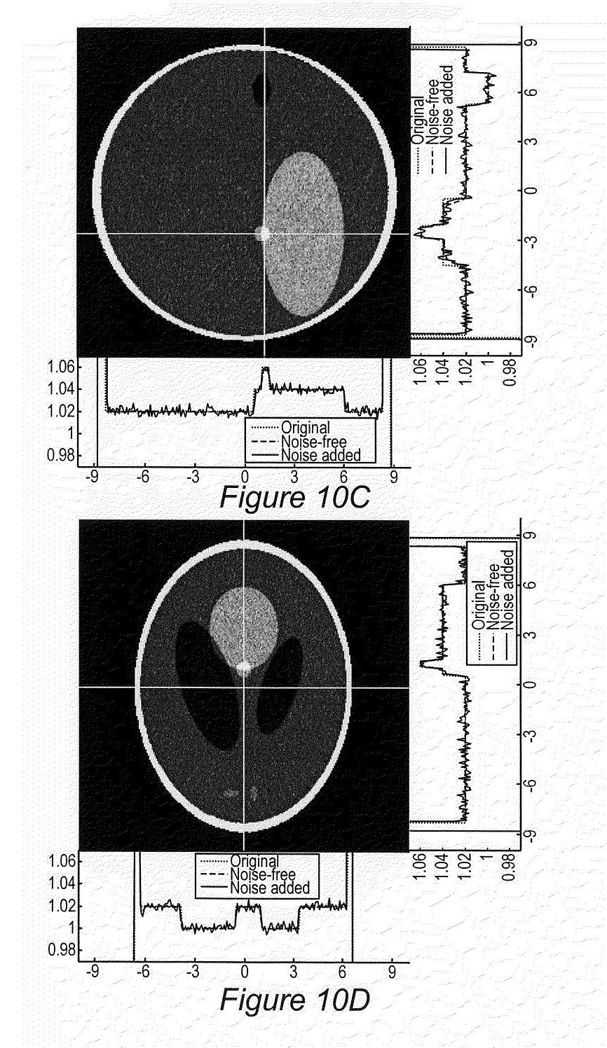

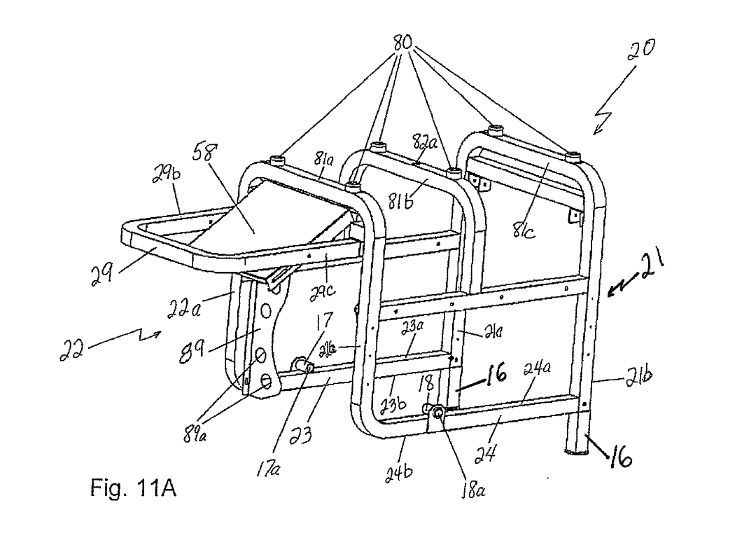

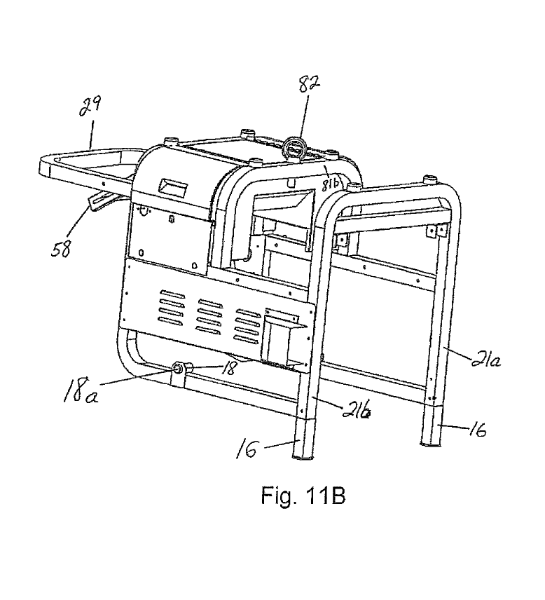

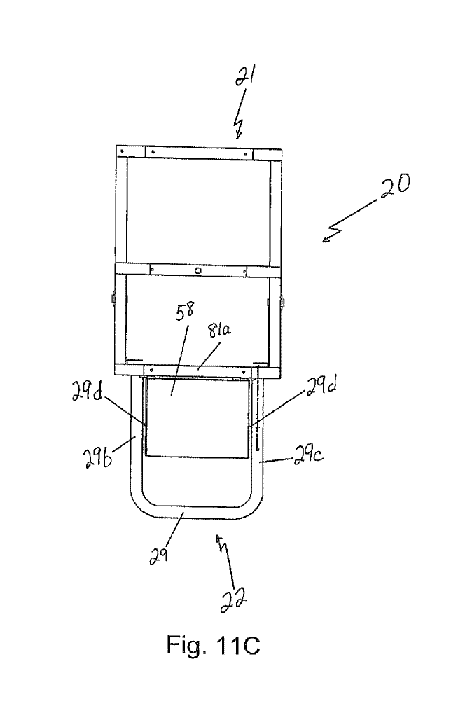

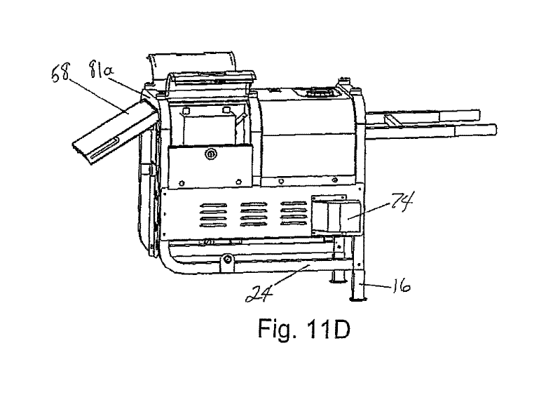

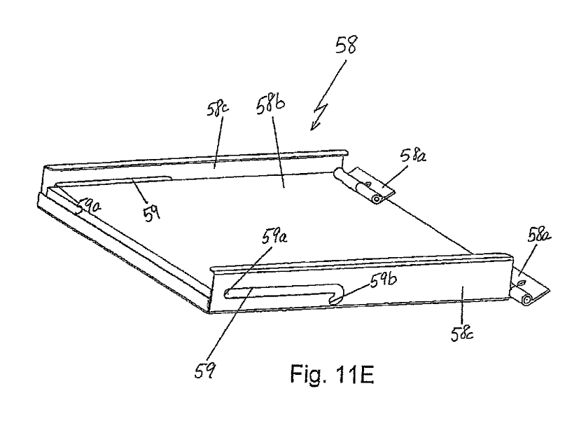

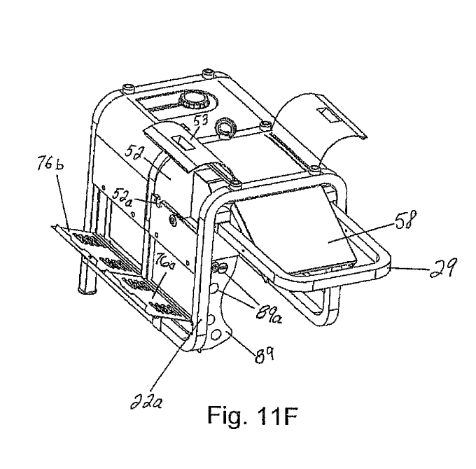

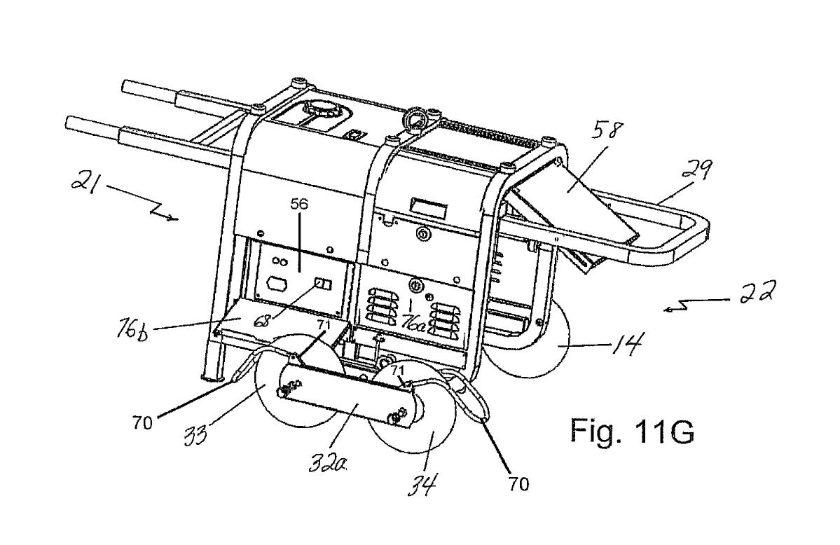

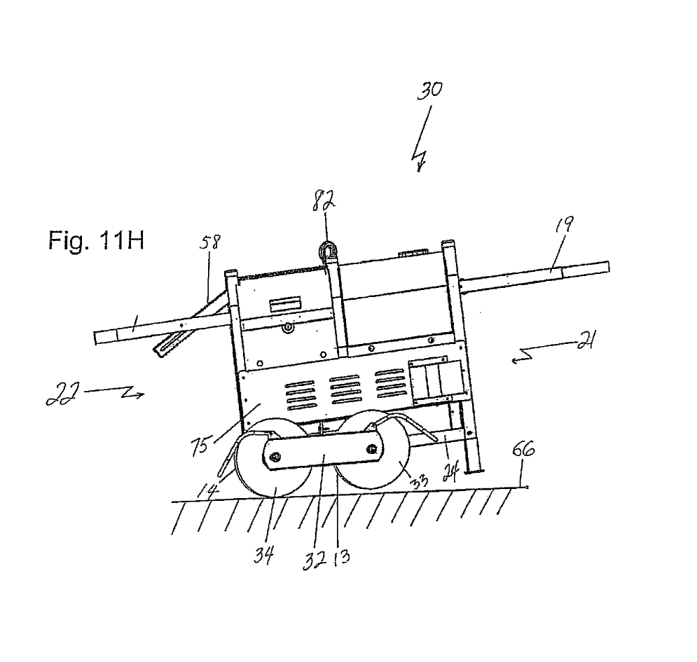

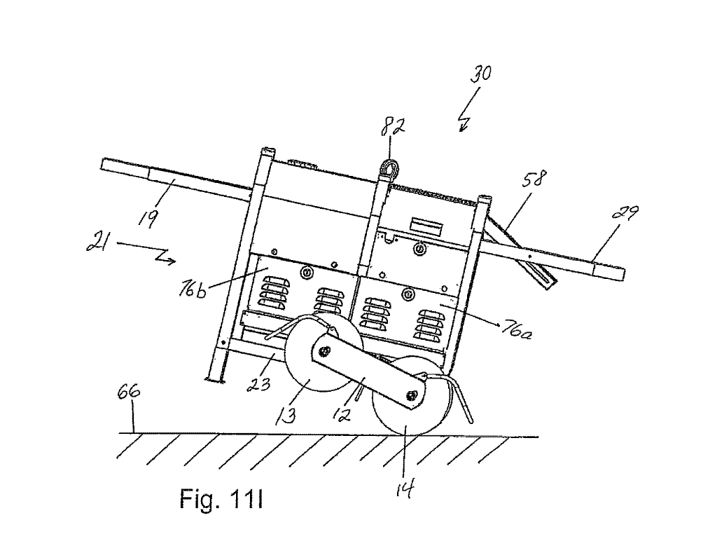

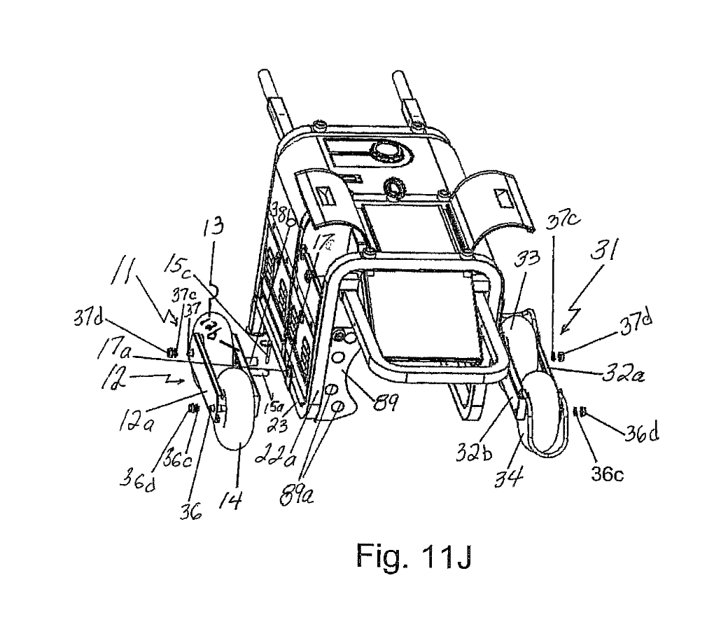

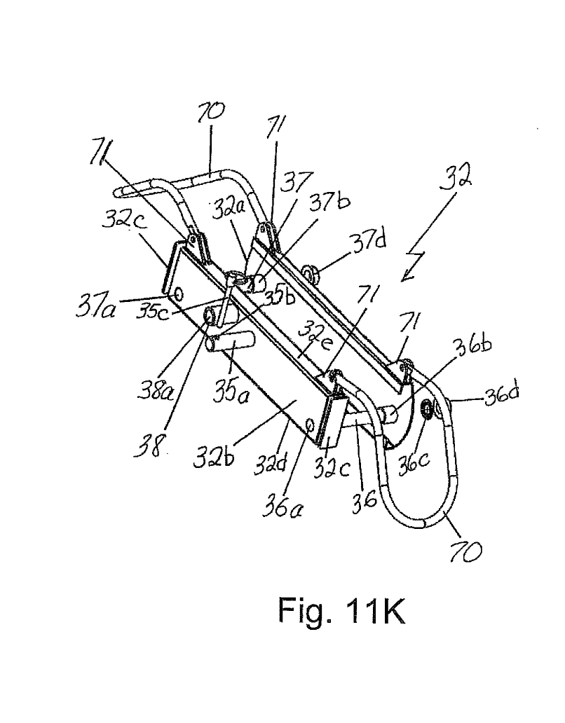

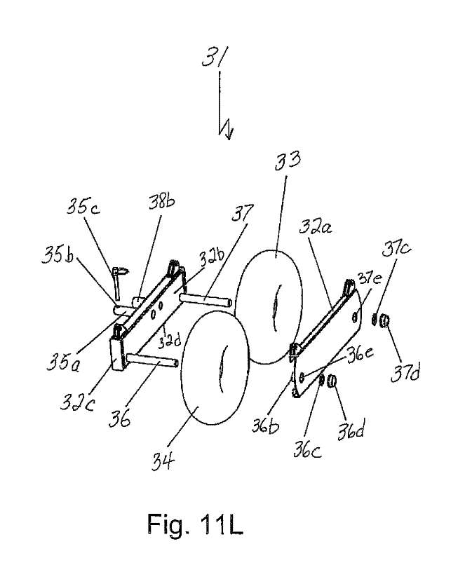

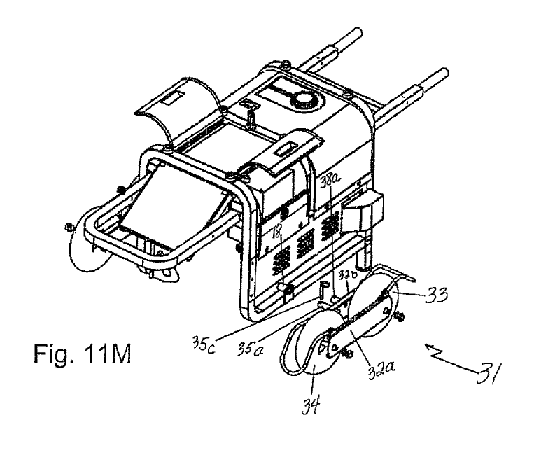

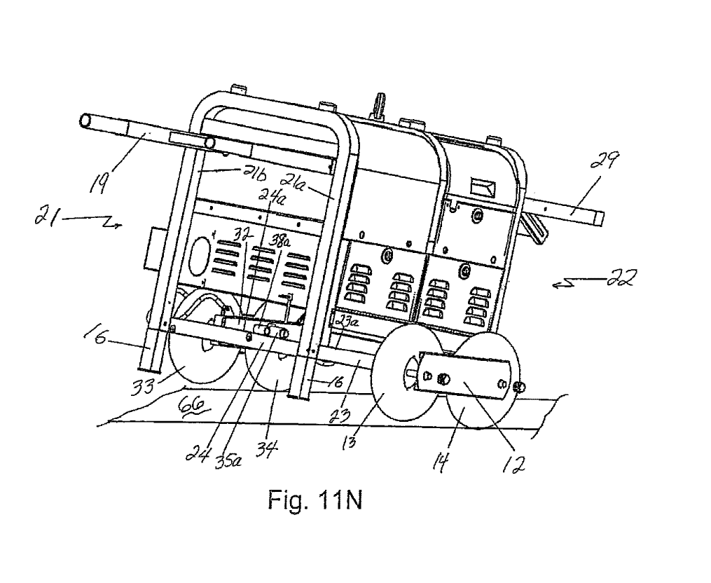

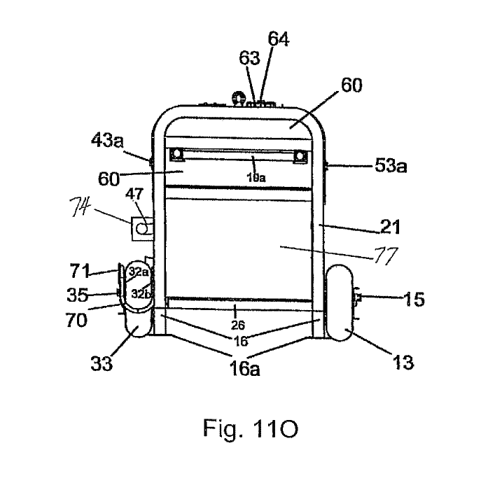

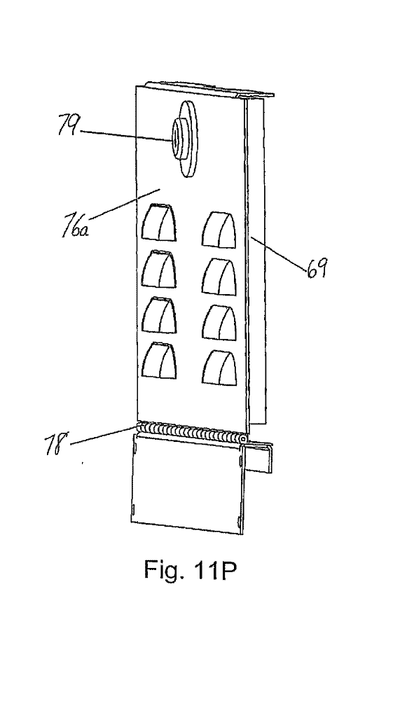

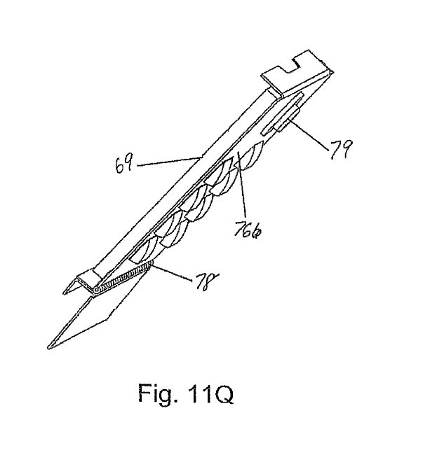

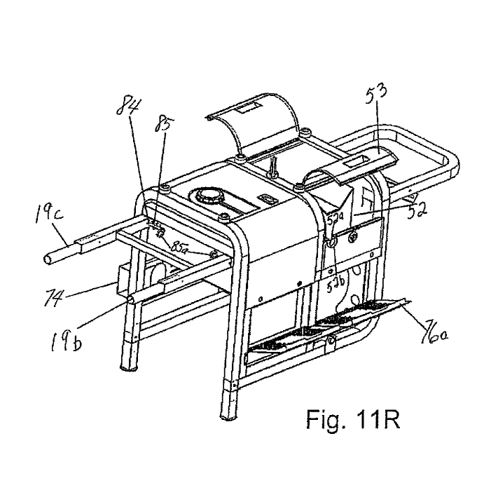

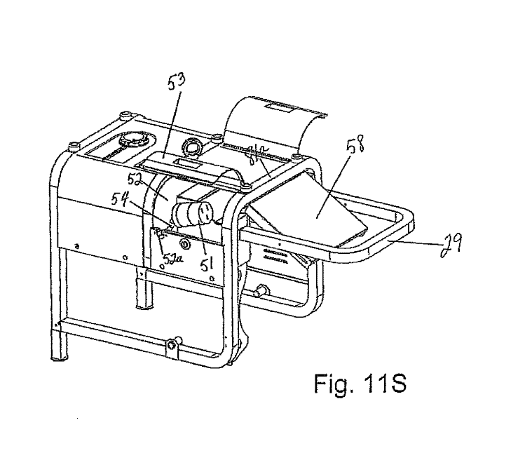

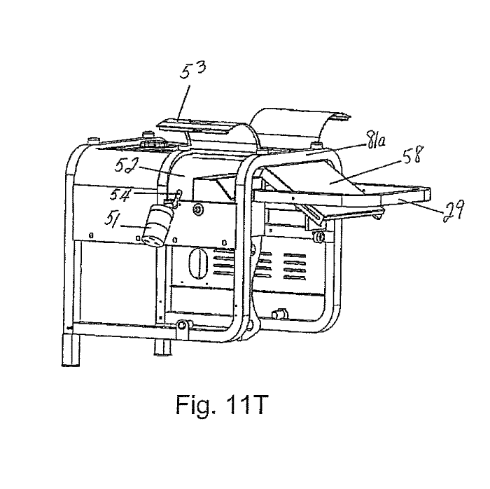

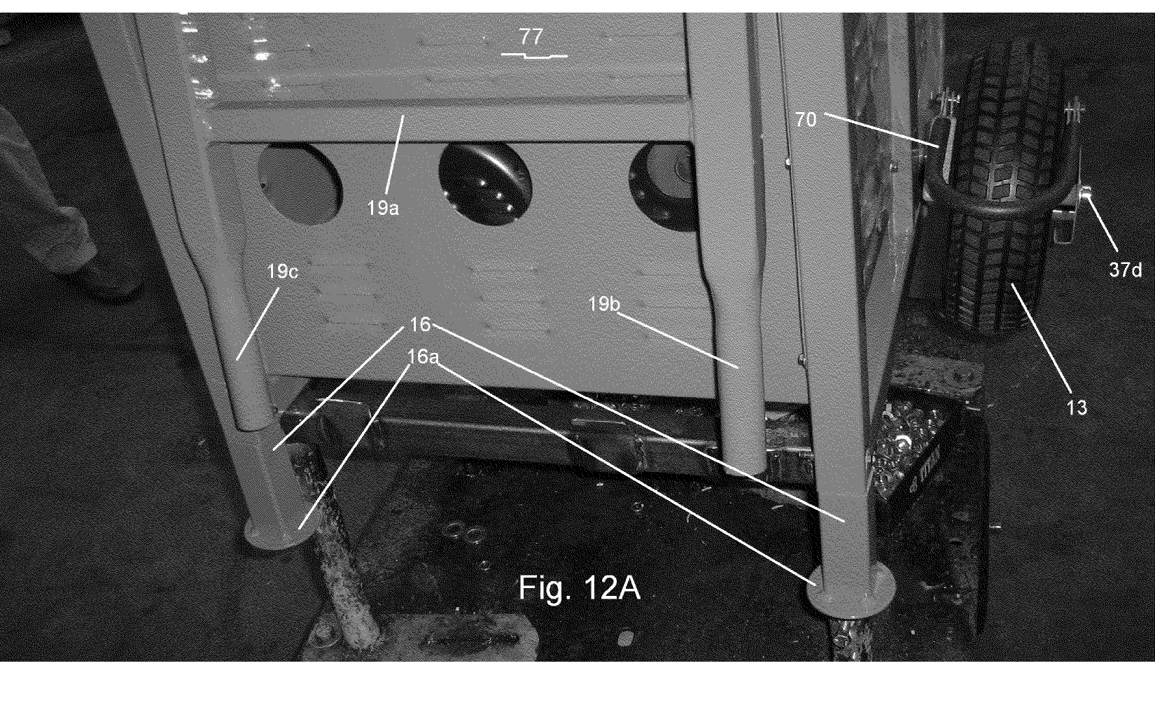

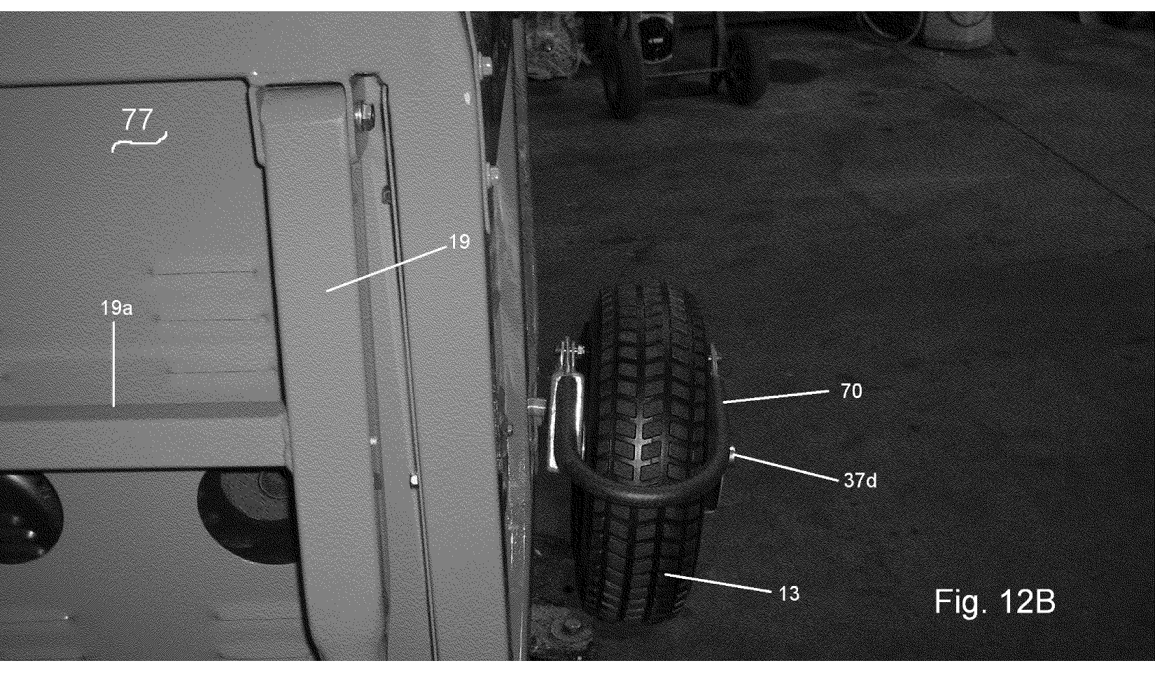

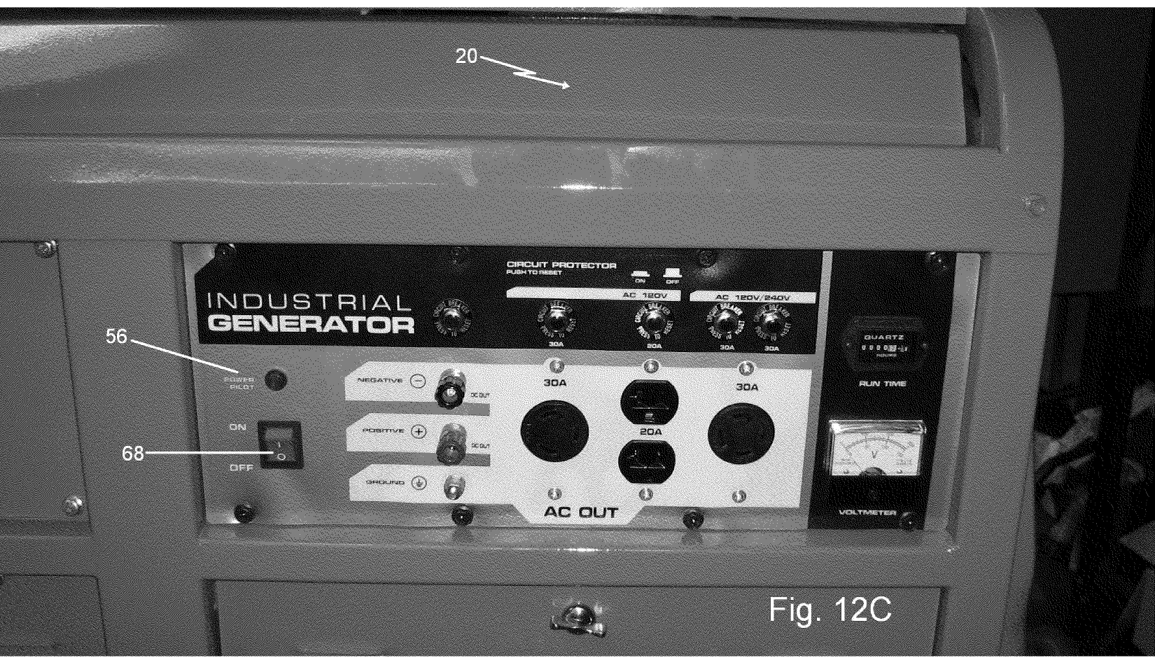

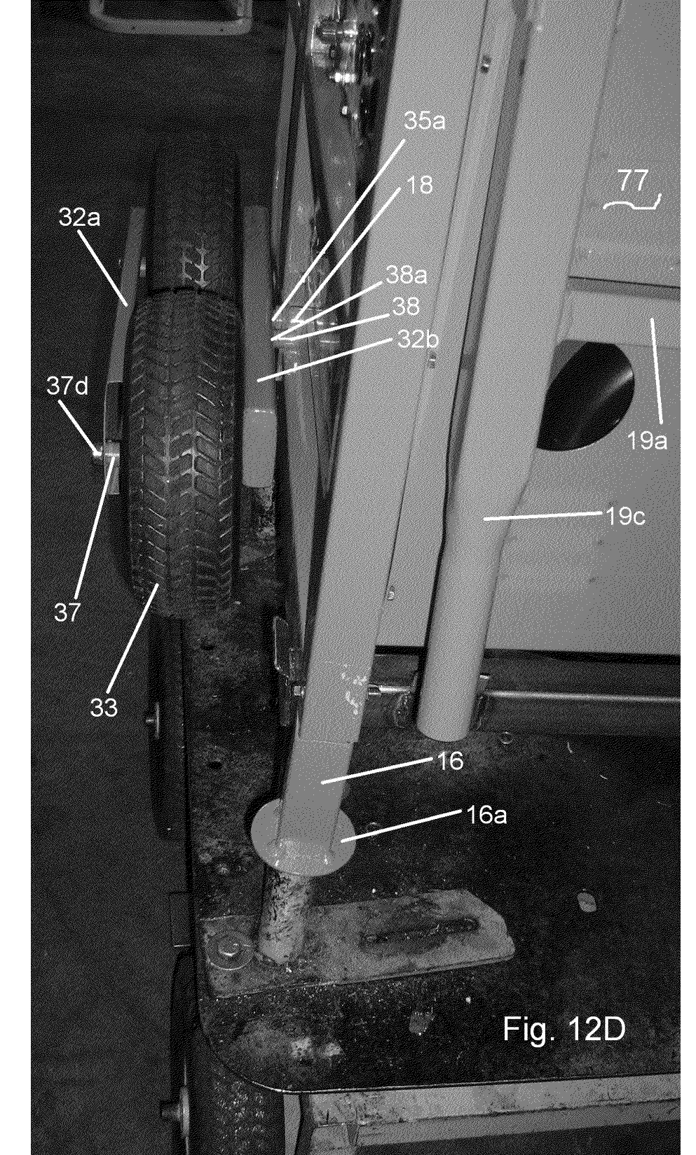

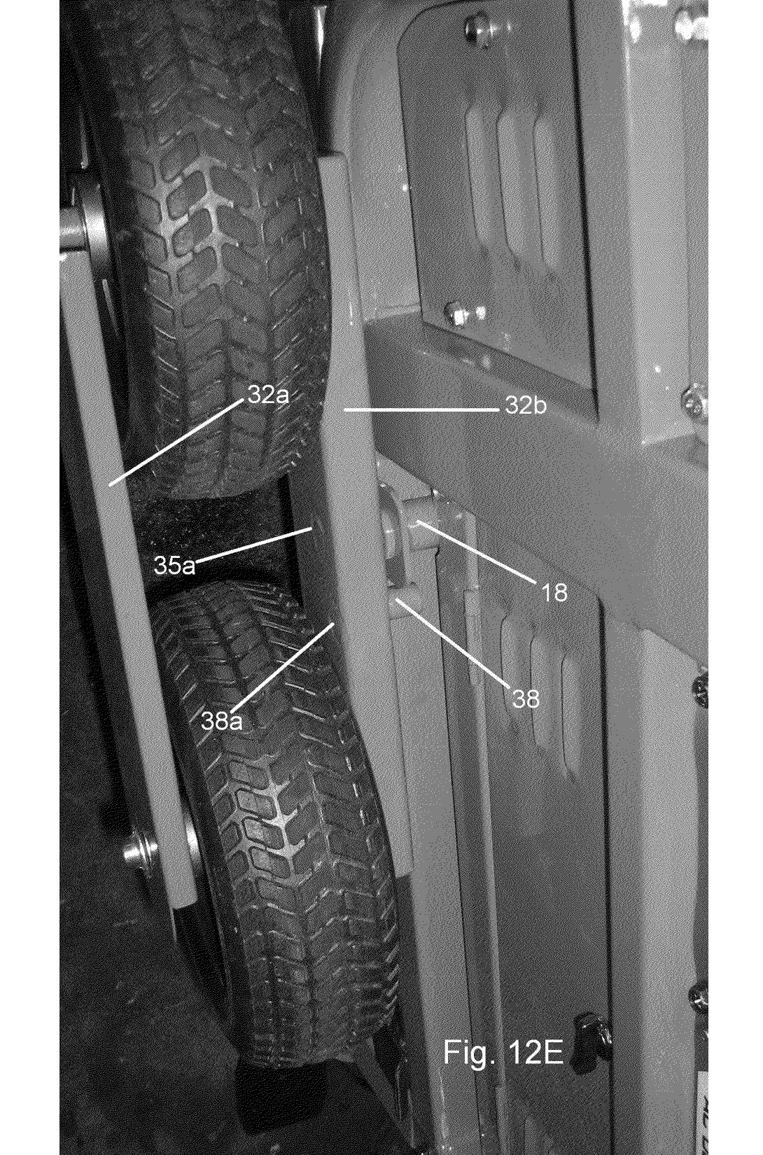

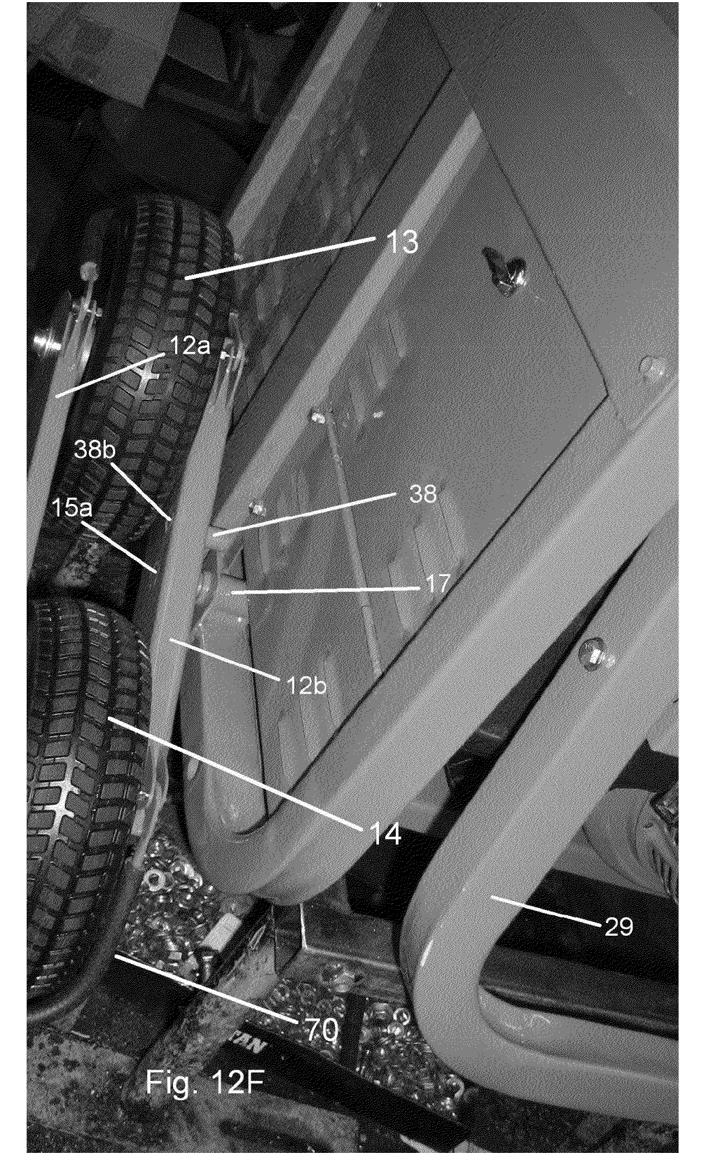

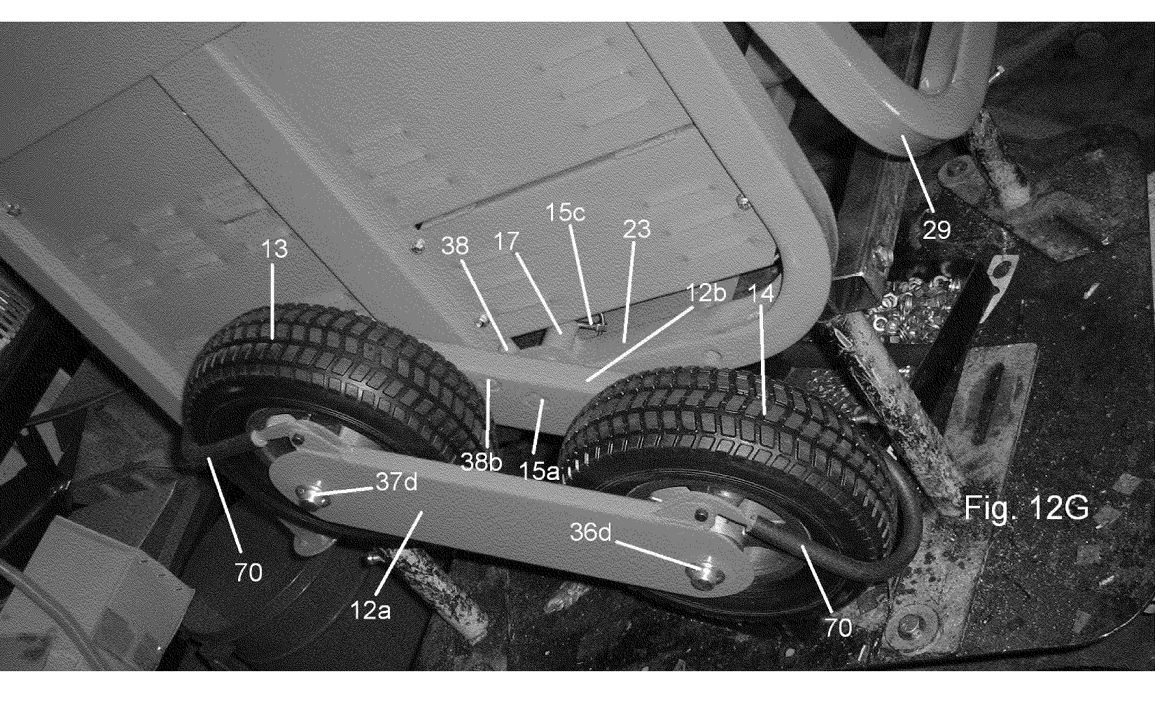

Similar to what we did for the reconstruction of a saddle curve, 258 starting points s_{b} were first uniformly selected from the interval [−0.4492π,−0.0208π]. From each μ(s_{b}), 545 chords were made with the end point parameter sₜ in the interval [s+0.88837π,s_{b}+1.1150π] uniformly. Furthermore, each chord contained 432 sampling points over a length 28.8 cm. Finally, the images were rebinned into a 256×256×256 matrix in the natural coordinate system. Both linear and bilinear interpolations were allowed in our implementation. Beside, our method was also evaluated with the noisy data by assuming that N₀ photons are emitted by the x-ray source. And only N photons arrive at the detector element after being attenuated in the object, and that the number of photons obeys a Poisson Distribution. The reconstructed noisy images were compared to their noise-free counterparts. The noise standard deviations in the reconstructed images were about 3.18×10⁻³ and 10.05×10⁻³ for N₀=10⁶ and 10⁵, respectively. FIGS. 9A to 9D and FIGS. 10A to 10D illustrate some typical image slices reconstructed from noise-free and noisy data, respectively. The slices shown in FIGS. 9A and 9B were reconstructed from noise-free data collected along the composite-circling trajectory, while the slices shown in FIGS. 9C and 9D were reconstructed from a saddle curve. We note that the strip artifacts in the reconstructed image (see FIG. 9B) were introduced by the interpolation at the projections of discontinuous phantom edge. These artifacts will disappear if we use a modified differentiable Shepp-Logan head phantom (see Yu,. H. Y., S. Y. Zhao, and G. Wang, “A differentiable Shepp-Logan phantom and its applications in exact cone-beam CT”, Physics in Medicine and Biology, 2005, 50(23): pp. 5583-5595).

To solve the reconstruction problem of a quasi-short object, we proposed a family of new saddle-like composite scanning mode. As a subset, the CCS mode has-been studied carefully, especially the case m=2. This does not mean that the case m=2 of the CCS mode is the optimal among the family of saddle-like curves. Our group members are working hard to investigate the properties of the saddle-like curves and optimize the configuration parameters. On the other hand, although the generalized BPF method has been developed to exact reconstruct images from data collected along a CCS trajectory, the method is not efficient because of its shift-variant property. Recently, Katsevich announced an important progress towards exact and efficient general cone-beam reconstruction algorithms for two classes of scanning loci (see Katsevich, A. and M. Kapralov, “Theoretically exact FBP reconstruction algorithms for two general classes of curves”, 9th International Meeting on Fully Three-Dimensional Image Reconstruction in Radiology and Nuclear Medicine, 2007, pp. 80-83, Lindau, Germany). The first class curves are smooth and of positive curvature and torsion. The second class consists of generalized circle-plus curves (see Katsevich, A., “Image reconstruction for a general circle-plus trajectory”, Inverse Problems, 2007, 23(5): pp. 2223-2230).

Regarding the engineering implementation of our composite-scanning mode, we recognize that the collimation problem must be effectively addressed. Because the x-ray source, detector array and collimators are mounted on the same data acquisition system (DAS), we can omit the rotation of the whole DAS. That is, the focal spot is circularly rotated in the plane parallel to the patient motion direction, and we need have a collimation design to reject most of scattered photons for any focal spot position. During the scan, we can adjust the direction and position of the detector array and associated collimators to keep the line connecting the detector array center and the focal spot perpendicular to the detector plane and make all the collimators focus on the focal spot all the time. This can be mechanically done, synchronized by the rotation of the focal spot. In this case, the focal spot rotation plane and the detector plane are not parallel in general. Other designs for the same purpose are possible in the same spirit of this invention. Furthermore, our approach can also be adapted for inverse geometry based cone-beam CT.

In conclusion, we have developed a new CCS mode for the quasi-short problem, which has better mechanical rotation stability and physiological condition compatibility because of its symmetry. The generalized BPF method has been developed to reconstruct image from data collected along a CCS trajectory for the case m=2. The initial simulation results have demonstrated the merits of the proposed CCS mode and validate the correctness of the exact reconstruction algorithm.

### Appendix A. Derivative of Formulae (Eqs. 10-11)

For a given unit direction p, its projection position in the local coordinate system can be expressed as:

$$\begin{matrix}u = \frac{D β \cdot {d}_{1}}{β \cdot {d}_{3}} + {R}_{1 b} sin ( ms ) , & ( \text{A-1a} ) \\ v = \frac{D β \cdot {d}_{2}}{β \cdot {d}_{3}} + {R}_{1 a} cos ( ms ) \cdot & ( \text{A-1b} )\end{matrix}$$

Hence, we have

$$\begin{matrix}\begin{matrix}\frac{\partial u}{\partial s} = ( \frac{D β \cdot {d}_{1}}{β \cdot {d}_{3}} ) \\ = \frac{D β \cdot {d}_{1}}{β \cdot {d}_{3}} - \frac{D β \cdot {d}_{1} β \cdot {d}_{3}}{{( β \cdot {d}_{3} )}^{2}} + {mR}_{1 b} cos ( ms ) ,\end{matrix} & ( \text{A-2a} ) \\ \begin{matrix}\frac{\partial v}{\partial s} = ( \frac{D β \cdot {d}_{2}}{β \cdot {d}_{3}} ) \\ = \frac{D β \cdot {d}_{2}}{β \cdot {d}_{3}} - \frac{D β \cdot {d}_{2} β \cdot {d}_{3}}{{( β \cdot {d}_{3} )}^{2}} - {mR}_{1 a} sin ( ms ) \cdot \end{matrix} & ( \text{A-2b} )\end{matrix}$$

Noting d₁′=d₃,d₂′=0 and d₃′=−d₁, we obtain

$$\begin{matrix}\frac{\partial u}{\partial s} = \frac{D β \cdot {d}_{3}}{β \cdot {d}_{3}} + \frac{{D ( β \cdot {d}_{1} )}^{2}}{{( β \cdot {d}_{3} )}^{2}} + {mR}_{1 b} cos ( ms ) , & ( \text{A-3a} ) \\ \frac{\partial v}{\partial s} = \frac{D β \cdot {d}_{2} β \cdot {d}_{1}}{{( β \cdot {d}_{3} )}^{2}} - {mR}_{1 a} sin ( ms ) \cdot & ( \text{A-3b} )\end{matrix}$$

Using (A-1), it follows readily that

$$\begin{matrix}\frac{\partial u}{\partial s} = \frac{{( u - {R}_{1 b} sin ( ms ) )}^{2}}{D} + D + {mR}_{1 b} cos ( ms ) , & ( \text{A-4a} ) \\ \frac{\partial v}{\partial s} = \frac{( u - {R}_{1 b} sin ( ms ) ) ( v - {R}_{1 a} cos ( ms ) )}{D} - {mR}_{1 a} sin ( ms ) \cdot & ( \text{A-4b} )\end{matrix}$$

### Appendix B. Proof of the Convex Projection Condition R_{1b}≦R₂/(2m)

The projection of our CCS trajectory in the x-y plane can be expressed as

P_{Γ}={ρ(s)|ρ₁(s)=R₂ cos(s)−R_{1b} sin(ms)sin(s),ρ₂(s)+R_{1b} sin(ms)cos(s)} (B-1)

According to Liu and Traas (Lemma 2.7), a single closed regular C²-continuous curve is globally convex if and only if the curvature at every point on the curve is non-positive (see Liu, C. and C. R. Traas, “On convexity of planar curves and its application in CAGD”, Computer Aided Geometric Design, 1997, 14(7): pp. 653-669). Hence, it is required to satisfy ρ′(s)×ρ″(s)≧0 for any s ∈. Noting that

$$\begin{matrix}\{ \begin{matrix}{ρ}_{1}^{′} ( s ) = - {R}_{2} sin ( s ) - {R}_{1 b} sin ( ms ) cos ( s ) - {mR}_{1 b} cos ( ms ) sin ( s ) \\ {ρ}_{2}^{′} ( s ) = {R}_{2} cos ( s ) - {R}_{1 b} sin ( ms ) sin ( s ) + {mR}_{1 b} cos ( ms ) cos ( s )\end{matrix} , \\ And & ( \text{B-2} ) \\ \{ \begin{matrix}{ρ}_{1}^{″} ( s ) = - {R}_{2} cos ( s ) + {R}_{1 b} ( {m}^{2} + 1 ) sin ( ms ) sin ( s ) - 2 {mR}_{1 b} cos ( ms ) cos ( s ) \\ {ρ}_{2}^{″} ( s ) = - {R}_{2} sin ( s ) - {R}_{1 b} ( {m}^{2} + 1 ) sin ( ms ) cos ( s ) - 2 {mR}_{1 b} cos ( ms ) sin ( s )\end{matrix} & ( \text{B-3} )\end{matrix}$$

there will be

$${ρ}^{′} ( s ) \times {ρ}^{″} ( s ) = {ρ}_{1}^{′} ( s ) {ρ}_{2}^{″} ( s ) - {ρ}_{1}^{″} ( s ) {ρ}_{2}^{′} ( s ) = ( {R}_{2} sin ( s ) + {R}_{1 b} sin ( ms ) cos ( s ) + {mR}_{1 b} cos ( ms ) sin ( s ) ) \times ( {R}_{2} sin ( s ) + {R}_{1 b} ( {m}^{2} + 1 ) sin ( ms ) cos ( s ) + 2 {mR}_{1 b} cos ( ms ) sin ( s ) ) + ( {R}_{2} cos ( s ) - {R}_{1 b} ( {m}^{2} + 1 ) sin ( ms ) sin ( s ) + 2 {mR}_{1 b} cos ( ms ) cos ( s ) ) \cdot \times ( {R}_{2} cos ( s ) - {R}_{1 b} sin ( ms ) sin ( s ) + {mR}_{1 b} cos ( ms ) cos ( s ) ) = ( {m}^{2} - 1 ) {R}_{1 b}^{2} {cos}^{2} ( ms ) + 3 {mR}_{2} {R}_{1 b} cos ( ms ) + ( {m}^{2} + 1 ) {R}_{1 b}^{2} + {R}_{2}^{2} \qquad ( \text{B-4} )$$

Denote z=tg²(ms/2), we arrive at

$${ρ}^{′} ( s ) \times {ρ}^{″} ( s ) \geq 0 \\ ⇔ ( {m}^{2} - 1 ) {{R}_{1 b}^{2} ( \frac{1 - z}{1 + z} )}^{2} + 3 {mR}_{2} {R}_{1 b} ( \frac{1 - z}{1 + z} ) + ( {m}^{2} + 1 ) {R}_{1 b}^{2} + {R}_{2}^{2} \geq 0 \\ ⇔ ( {R}_{2}^{2} + 2 {m}^{2} {R}_{1 b}^{2} - 3 {mR}_{2} {R}_{1 b} ) {z}^{2} + 2 ( {R}_{2}^{2} + 2 {R}_{1 b}^{2} ) z + ( {R}_{2}^{2} + 2 {m}^{2} {R}_{1 b}^{2} + 3 {mR}_{2} {R}_{1 b} ) \geq 0 , \qquad ( \text{B-5} )$$

where the relationship

$$cos ( ms ) = \frac{1 - z}{1 + z}$$

has been used. Noticing the facts R₂>0, R_{1b}≧0, 2(R₂²+2R^{2′}_{1b})>0 and (R₂²+2m²R²_{1b}+3mR₁R_{1b})>0, we get the necessary and sufficient condition for ρ′(s)×ρ″(s)≧0 at any s ∈ as,

R₂²+2m²R²_{1b}−3mR₂R_{1b}≧0, (B-6)

which implies that R_{1b}R₂/(2m) or R_{1b}≧R₂/m. When R_{1b}≧R₂/m, the curve P_{Γ} becomes a complex curve (not single) which should be omitted. Hence, R_{1b}≧R₂/(2m) is the necessary and sufficient condition for the convex projection of the CCS trajectory in the x-y plane.

While the invention has been described in terms of a single preferred embodiment, those skilled in the art will recognize that the invention can be practiced with modification within the spirit and scope of the appended claims.

Having thus described our invention, what we claim as new and desire to secure by Letters Patent is as follows:

1. A method of composite-circling scanning (CCS) mode for computed tomography (CT) comprising the steps of:
rotating an x-ray focal spot of an x-ray source along a circular trajectory on a plane facing an object to be reconstructed;
simultaneously rotating the x-ray source around the object in a circular trajectory on a gantry encircling the object;
acquiring a dataset resulting from the composite scanning mode; and
mathematically reconstructing an image of the object using a computer.

2. The method of claim 1, wherein the composite-circling scanning (CCS) mode is a composite scanning mode wherein an x-ray focal spot moves on a plane facing an object to be reconstructed.

3. The method of claim 1, wherein the rotation of the x-ray source around the object is performed around a Z-axis passing through the object.

4. The method of claim 3, wherein the Z-axis is horizontal, parallel to the earth surface.

5. The method of claim 3, wherein the Z-axis is vertical, perpendicular to the earth surface.

6. The method of claim 1, further comprising the step of translating the object through the gantry while rotating the x-ray source around the object in a circular trajectory.

7. A composite-circling scanning (CCS) mode computed tomography (CT) system comprising:
an x-ray source;
a gantry encircling an object to be reconstructed and supporting the x-ray source for rotation about the object;
x-ray detectors mounted on the gantry opposite the x-ray source for rotation about the object;
means for rotating an x-ray focal spot on a plane facing the object;
means for simultaneously moving the x-ray source and the x-ray detectors on the gantry so as to rotate the x-ray source and the x-ray detectors around the object in a circular trajectory;
means responsive to outputs of the x-ray detectors for acquiring a dataset resulting from the composite scanning mode; and
computing means for mathematically reconstructing an image of the object.

8. The composite-circling scanning (CCS) mode computed tomography (CT) system of claim 7, wherein the means for rotating an x-ray focal spot rotates the focal spot on a plane facing the object to be reconstructed.

9. The composite-circling scanning (CCS) mode computed tomography (CT) system of claim 7, wherein the rotation of the x-ray source around the object is performed around a Z-axis passing through the object.

10. The composite-circling scanning (CCS) mode computed tomography (CT) system of claim 9, wherein the Z-axis is horizontal, parallel to the earth surface.

11. The composite-circling scanning (CCS) mode computed tomography (CT) system of claim 9, wherein the Z-axis is vertical, perpendicular to the earth surface.

12. The composite-circling scanning (CCS) mode computed tomography (CT) system of claim 7, further comprising means for translating the object through the gantry while the x-ray source is rotated around the object in a circular trajectory.
